(19) United States
(12) Patent Application Publication
(10) Pub. No.: US 2023/0380812 A1
(43) Pub. Date: Nov. 30, 2023

(54) MEDICAL IMAGING METHOD, APPARATUS, AND SYSTEM

(71) Applicant: GE Precision Healthcare LLC, Wauwatosa, WI (US)

(72) Inventor: Siying Wang, Wuxi (CN)

(21) Appl. No.: 18/326,759

(22) Filed: May 31, 2023

(30) Foreign Application Priority Data
May 31, 2022 (CN) 202210605204.2

(51) Int. Cl. A61B 8/08; G06T 7/11

(52) U.S. Cl. CPC A61B 8/5207; A61B 8/485; G06T 7/11; A61B 8/5223

(57) ABSTRACT

Provided in the present invention are a medical imaging method, apparatus, and system according to various embodiments. According to an embodiment, the medical imaging method includes performing image segmentation of a medical image acquired from a current scan containing a heart region of an examined subject to determine a heart wall region in said medical image. The medical imaging method includes generating a local elastic image of said heart wall region. And the medical imaging method includes displaying said local elastic image at the position of said heart wall region in said medical image in an overlapping manner and in real-time.

### CROSS-REFERENCE TO RELATED APPLICATION

This application claims priority to Chinese patent application number 202210605204.2, filed on May 31, 2022, the entirety of which is incorporated herein by reference.

### TECHNICAL FIELD

Embodiments of the present invention relate to the technical field of medical devices, and in particular to a medical imaging method, apparatus, and system.

### BACKGROUND

Elastography is one of the topics of special interest in clinical research in recent years. Elastography provides information about the elasticity and stiffness of tissues, and is mainly applied in clinical diagnosis of diseases in soft tissue organs. Compared with anatomical images, elastography can provide additional diagnostic information of tissue mechanical conditions, which can guide biopsy, and sometimes, when combined with other examinations, it can replace biopsy. For example, patients with liver diseases such as liver fibrosis and fatty liver disease usually have stiffer liver tissue than is found in normal livers. Elastography offers tremendous advantages in the diagnosis of liver diseases. The methods of elastography include ultrasound elastography, quasi-static elastography/strain imaging, magnetic resonance elastography, and so on.

### SUMMARY

According to an embodiment, a medical imaging method includes performing image segmentation of a medical image acquired from a current scan containing a heart region of an examined subject to determine a heart wall region in the medical image. The method includes generating a local elastic image of the heart wall region and displaying the local elastic image at the position of the heart wall region in the medical image in an overlapping manner and in real-time, wherein the local elastic image of the heart wall region is displayed only at the position of said heart wall region determined by the image segmentation.

According to an embodiment, a medical imaging apparatus includes a segmentation unit which performs image segmentation of a medical image acquired from a current scan containing a heart region of an examiner subject to determine a heart wall region in the medical image. The medical imaging apparatus includes a generation unit which generates a local elastic image of the heart wall region and a display unit which displays the local elastic image at the position of the heart wall region in the medical image in an overlapping manner and in real-time, wherein the local elastic image of the heart wall region is displayed only at the position of said heart wall region determined by the image segmentation.

According to an embodiment, a medical imaging system includes a scan device which is used to scan a heart region of an examined subject to obtain imaging data. The medical imaging system includes a processor which is configured for generating a medical image containing the heart of the medical image to determine a heart wall region in the medical image. The processor is configured to generate a local elastic image of the heart wall region. The medical imaging system includes a display which displays the local elastic image at the position of the heart wall region in the medical image in an overlapping manner and in real-time, wherein the local elastic image of the heart wall region is displayed only at the position of said heart wall region determined by the image segmentation.

One of the benefits of the embodiments of the present invention is that: an elastic imaging is displayed only in the heart wall region in an overlapping manner and in real-time by segmenting the heart wall region, whereby the elastic imaging of the heart wall region can be more intuitively observed, which allows for real-time assessment of myocardial strain, and helps in rapid clinical diagnosis.

With reference to the following description and accompanying drawings, specific embodiments of the examples of the present application are disclosed in detail, and manners in which the principle of the examples of the present application is employed are illustrated. It should be understood that the embodiments of the present application are not thereby limited in scope. Within the spirit and scope of the appended claims, the embodiments of the present application comprise various changes, modifications, and equivalents.

### BRIEF DESCRIPTION OF THE DRAWINGS

The accompanying drawings are included to provide further understanding of embodiments of the present application, constitute a part of the specification, and are used to illustrate embodiments of the present application and set forth the principles of the present application together with textual description. Obviously, the accompanying drawings in the following description are merely some embodiments of the present application, and a person of ordinary skill in the art may obtain other embodiments according to the accompanying drawings without the exercise of inventive effort. In the accompanying drawings:

FIG. 1 is a schematic diagram of a medical imaging method according to an embodiment of the present application;

FIG. 2 is a schematic diagram of a medical image according to an embodiment of the present application;

FIG. 3 is a schematic diagram of a heart wall region according to an embodiment of the present application;

FIG. 4 is a schematic diagram of a heart wall region according to an embodiment of the present application;

FIG. 5 is a schematic diagram of a method for generating a local elastic image according to an embodiment of the present application;

FIG. 6 is a schematic diagram of a local elastic image according to an embodiment of the present application;

FIG. 7 is a schematic diagram showing a medical image and a local elastic image displayed in an overlapping manner according to an embodiment of the present application;

FIG. 8 is a schematic diagram of a medical imaging apparatus according to an embodiment of the present application;

FIG. 9 is a schematic diagram of a generation unit according to an embodiment of the present application;

FIG. 10 is a schematic diagram of a medical imaging system according to an embodiment of the present application;

FIG. 11 is a schematic diagram of an ultrasound imaging system according to an embodiment of the present application.

### DETAILED DESCRIPTION

The foregoing and other features of the embodiments of the present application will become apparent from the following description with reference to the accompanying drawings. In the description and the accompanying drawings, specific embodiments of the present application are specifically disclosed, and part of the embodiments in which the principles of the examples of the present application may be employed are indicated. It should be understood that the present application is not limited to the described embodiments. On the contrary, the embodiments of the present application include all modifications, variations, and equivalents falling within the scope of the appended claims.

The features described and/or illustrated for one embodiment may be used in one or more other embodiments in the same or similar manner, combined with features in other embodiments, or replace features in other embodiments. The term “include/comprise” when used herein refers to the presence of features, integrated components, steps, or assemblies, but does not preclude the presence or addition of one or more other features, integrated components, steps, or assemblies.

Currently, elastography is increasingly used in cardiac diagnosis. In the existing methods, strain can be calculated by pre-acquiring multiple frames of B-mode scanned images and by speckle tracking to assess whether the heart is diseased. However, the above method requires pre-acquisition of multiple frames of scanned images and is therefore only suitable for off-line processing and cannot acquire strain in the heart wall region in real-time. In addition, there are some methods of real-time elastography in the prior art, but the aforementioned elastography cannot be localized to the heart region for real-time display.

In response to at least one of the above technical problems, embodiments of the present invention provide a medical imaging method, apparatus, and system. The following is a specific description of an embodiment of the present invention with reference to the accompanying drawings.

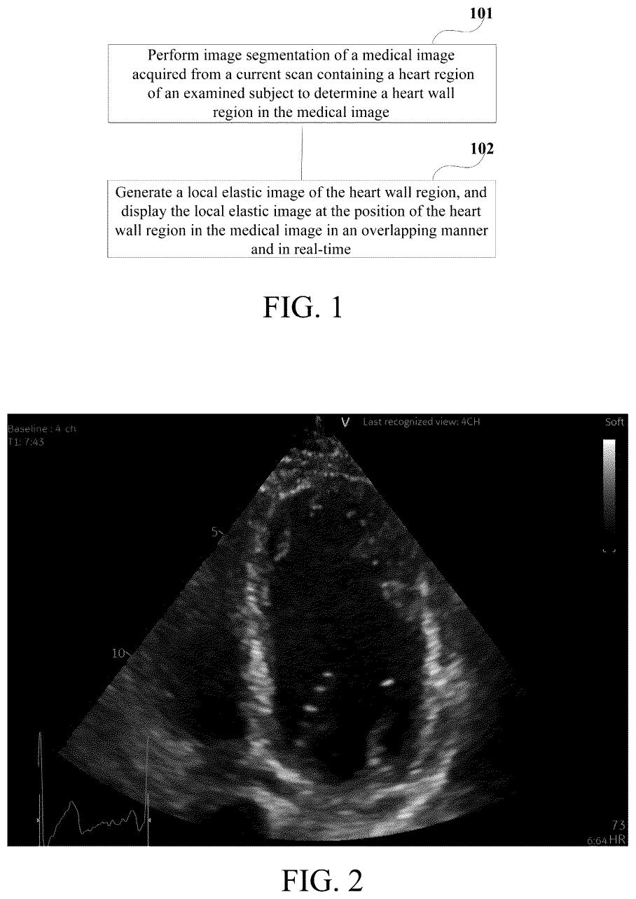

Embodiments of the present invention provide a method for medical imaging, and FIG. 1 is a schematic diagram of a medical imaging method according to an embodiment of the present application. As shown in FIG. 1, the medical imaging method includes step 101 and step 102. Step 101 includes performing image segmentation of the medical image acquired from a current scan containing a heart region of an examined subject to determine a heart wall region in the medical image. And step 102 includes generating a local elastic image of the heart wall region, and displaying the local elastic image at the position of the heart wall region in the medical image in an overlapping manner and in real-time.

In some embodiments, in step 101, the heart region includes at least one of the left ventricle, the right ventricle, the left atrium, and the right atrium, as illustrated below with the heart region being the left ventricle.

In some embodiments, the medical images may be acquired by various medical imaging modalities including, but not limited to: ultrasound imaging, fluoroscopy, Computed Tomography (CT), Magnetic Resonance Imaging (MRI), C-arm imaging, Positron Emission Computed Tomography (PET), Single Photon Emission Computed Tomography (SPECT), or any other suitable medical imaging techniques.

In some embodiments, the medical image can be a two-dimensional image or a three-dimensional image or a four-dimensional image, which is obtained in real-time by any of the above medical imaging modalities. In the case of ultrasound imaging, for example, a real-time non-invasive high-frequency sound wave is emitted by a probe to the examined subject, the reflected imaging data are collected, and the corresponding medical image is generated in real-time. The medical image acquired by the current scan may refer to a medical image (anatomical image of a specific section) that can reflect the current state (morphology) of the organ or tissue (e.g., the heart) of the examined subject at the current time (in real-time).

In some embodiments, the medical image may be a grayscale image in order to facilitate overlay display of a local elastic graphic. For example, the medical image may be an ultrasound B-mode image, but embodiments of the present invention are not limited thereto.

In some embodiments, in step 101, image segmentation may be performed using deep learning algorithms. For example, the medical image is segmented using a deep neural network (e.g., a convolutional neural network) to determine the heart wall region in the medical image, e.g., the deep neural network may contain, for example, an input layer, an output layer, and one or more hidden layers between the input layer and the output layer. Each layer can consist of multiple processing nodes that can be referred to as neurons. For example, the input layer may have neurons for each pixel or set of pixels from the scan plane of the anatomical structure. The output layer may have neurons corresponding to a plurality of predefined structures or predefined types of structures (or organizations therein). Each neuron in each layer may perform processing functions and pass processed medical image information to one of a plurality of neurons in the downstream layer for further processing. For example, neurons in the first layer may learn to recognize structural edges in medical image data. Neurons in the second layer may learn to recognize shapes etc., based on the detected edges from the first layer.

For example, the deep neural network may use a U-Net network model, and the medical image is fed into the neural network model, and the output of the neural network model results in a segmentation of the heart wall region. The heart wall region is a region including heart muscles, and optionally, the heart wall region can further include the endocardium and/or epicardium, etc. The heart wall region in the segmentation result can be represented by the image after the boundary contour (which can also include the region within the contour) is marked, and the boundary contour (inner) marking is the marking of the boundary contour (inner) of the heart wall in the original medical image, which consists of feature points (pixel points). For example, the segmentation result may be a mask image containing boundary contour (inner) markers that are the same size as the original medical image, in which the pixel value at the pixel position corresponding to the position of the heart wall boundary contour in the original medical image is 1, and the pixel values of the other pixel positions are 0. Alternatively, in the mask image, the pixel values at the pixel position corresponding to the position of the heart wall boundary contour and the position within the contour in the original medical image are 1 and the pixel values at the other pixel positions are 0, and then the mask image is a 0-1 mask image.

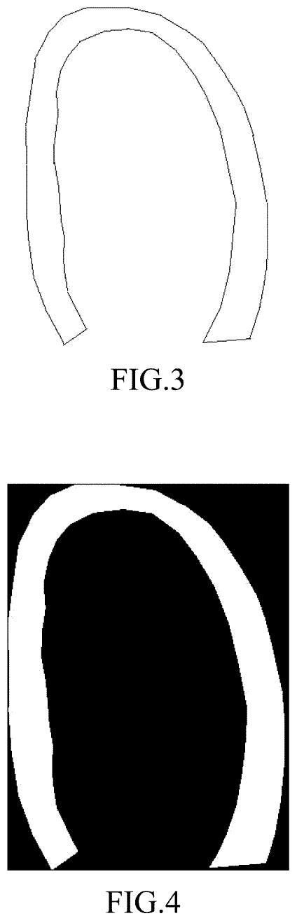

FIG. 2 is a schematic diagram of a medical image according to an embodiment of the present application, and FIGS. 3 and 4 are schematic diagrams of a heart wall region according to an embodiment of the present application. As shown in FIG. 2, the original medical image is an ultrasound image of the left ventricle. As shown in FIG. 3, the segmentation result is a left ventricular myocardial region (contour) obtained from segmentation, and as shown in FIG. 4, the segmentation result is a left ventricular myocardial region (on contour and within contour) obtained from segmentation.

In some embodiments, the method may optionally further include: training the neural network, for example, based on a known input data set (medical images) and a known output data set (e.g., a mask image by manually labeling medical images as described above) (image pairs). By setting the number of neurons in the neural network, and optimizing the network parameters (including but not limited to weights, biases, etc.) to identify the mathematical relationship between known inputs and desired outputs and the mathematical relationship for characterizing the inputs and outputs of each layer, the loss function converges, so as to train and obtain the aforementioned neural network. The loss function may be a cross-entropy function, but the embodiment of the present invention is not limited thereto.

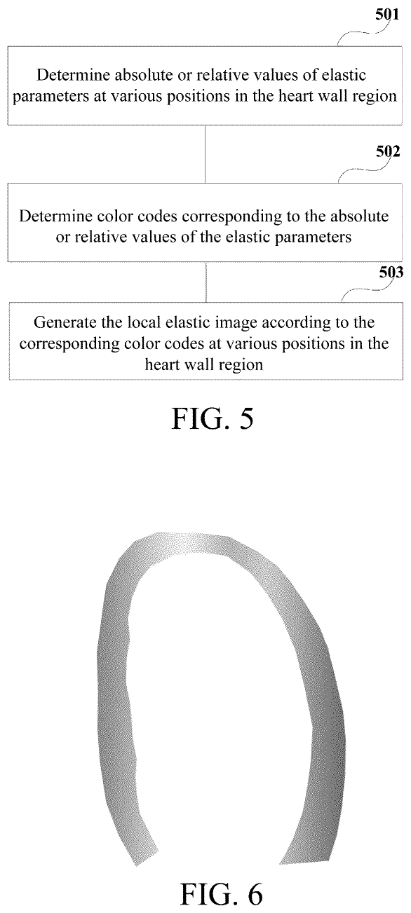

In some embodiments, after segmentation to obtain a heart wall region, at step 102, a local elastic image of the heart wall region is generated instead of an elastic image corresponding to the entire medical image. FIG. 5 is a schematic diagram of the method at step 102 for generating a local elastic image according to an embodiment of the present application. As shown in FIG. 5, step 102 includes steps 501, 502, and 503. At step 501, the method determines absolute or relative values of elastic parameters at various positions in the heart wall region. As step 502, the method determines color codes corresponding to the absolute or relative values of the elastic parameters. And, at step 503, the method generates the local elastic image according to the corresponding color codes at various positions in the heart wall region.

In some embodiments, the elastic parameter is a parameter that reflects the stiffness of the tissue organ and includes one of Young's modulus, elastic modulus, shear modulus, and shear wave propagation velocity. However, the present invention is not limited thereto, and the elastic parameter may also be referred to as strain, or stiffness, or hardness. The absolute value of the elastic parameter at each position in the heart wall region can be the absolute value of Young's modulus, the absolute value of elastic modulus, the absolute value of shear modulus, or the absolute value of shear wave propagation velocity at each position in the heart wall region. The relative value of the elastic parameter at each position in the heart wall region (also referred to as strain rate, or hardness ratio, or stiffness ratio) can be the ratio of Young's modulus of the heart wall region to the Young's modulus of the reference tissue, the ratio of the elastic modulus of the heart wall region to the elastic modulus of the reference tissue, the ratio of the shear modulus of the heart wall region to the shear modulus of the reference tissue, or the ratio of the shear wave propagation velocity of the heart wall region to the shear wave propagation velocity of the reference tissue.

In some embodiments, the absolute or relative values of the elastic parameters at various positions in the heart wall region can be determined using existing elastography techniques, e.g., if the medical image is obtained by ultrasound imaging, the absolute or relative values of the elastic parameters can be determined using strain-based ultrasound elastography, or shear-wave ultrasound elastography. If the medical image is obtained by magnetic resonance imaging, the absolute or relative values of the elastic parameters can be determined using magnetic resonance elastography. There is an approximate relationship between shear wave propagation velocity and elastic modulus, Young's modulus, and shear modulus: E=3pc²=3G, wherein c denotes the shear wave velocity, p denotes the tissue density, E denotes the Young's modulus value of the tissue, and G denotes the shear (elastic) modulus value of the tissue.

For example, strain-based ultrasound elastography produces a certain deformation mainly by pressing the tissue with an ultrasonic probe. Since the exact value of the external force is not known, the absolute values of the tissue elastic parameters cannot be measured quantitatively, but the relative values of the tissue elastic parameters can be calculated by comparing the degree of deformation of different tissues in the imaging area. For example, the amount of displacement occurring in the corresponding positions of different tissues before and after deformation is calculated, e.g., the ratio of Young's modulus in the heart wall region relative to the Young's modulus of the reference tissue, and the ratio of elastic modulus in the heart wall region relative to the elastic modulus of the reference tissue are calculated, and the reference tissue may be fat.

For example, shear wave-based elastography reflects stiffness differences between tissues mainly by generating shear wave propagation within the tissue and detecting the propagation parameters (e.g., shear modulus or velocity) for imaging, allowing quantitative measurement of absolute values of tissue elastic parameters. For example, ultrasound waves are transmitted to the heart region of the examined subject and the shear waves propagating in the heart region is tracked, ultrasound echoes are received, and then Young's modulus or shear wave velocity in the heart wall region are calculated based on the ultrasound echo data.

For example, magnetic resonance elastography is performed by means of slight mechanical vibrations (between 30 and 70 Hz) propagating through an external vibrating device to the region of the tissue to be studied, and the dynamic propagation of the vibrational waves within the tissue is captured by a magnetic resonance imaging machine (MRI). In post-processing, the absolute values of the elastic parameters of the tissue, such as the absolute values of Young's modulus or the absolute values of the elastic modulus at various positions in the heart wall region, can be calculated based on the appearance of the vibration waves inside the tissue (wavelength and amplitude).

In some embodiments, in step 501, only the absolute or relative values of the elastic parameters at various (pixel) positions in the heart wall region may be determined directly. Alternatively, the absolute or relative values of the elastic parameters at various (pixel) positions in the entire region of the medical image may be determined firstly, and then, in combination with the heart wall region determined in step 101, the absolute or relative values of the elastic parameters at various (pixel) positions in the heart wall region are obtained by performing filtering on the basis of the absolute or relative values of the elastic parameters at various (pixel) positions in the entire region, e.g., by multiplying the absolute or relative values of the elastic parameters at various (pixel) positions in the entire region by the aforementioned mask image to obtain the absolute or relative values of the elastic parameters at various (pixel) positions in the heart wall region.

In some embodiments, the color codes corresponding to the range of values of the elastic parameters are determined. For example, different colors (and their hues) may be used to indicate soft and/or hard tissue areas, with different absolute or relative values of the elastic parameters corresponding to different colors. Tissue areas with higher absolute or relative values of the elastic parameters (softer) may be coded as red (with gradually increasing color saturation), while tissue areas with lower absolute or relative values of the elastic parameters (stiffer) are coded as blue (with gradually increasing color saturation). A local elastic image is generated according to the corresponding color codes at various positions in the heart wall region. The local elastic image is a color image, which corresponds to the medical image, and the local elastic image can be a two-dimensional image, or a three-dimensional image, or a four-dimensional image. The pixel value (ARGB value) at each pixel position (a position where the pixel value in the mask image is 1) in the heart wall region in the local elastic image is the corresponding color code value. The transparency A in the pixel value (ARGB value) at the pixel position other than the heart wall region is set to 0, or the pixel value (RGB value) is set to white, and FIG. 6 is a schematic diagram of a local elastic image according to an embodiment of the present application.

The above implementation method in FIG. 5 is only an example of an embodiment of step 102, and the present invention is not limited thereto. For example, it is also possible to generate an elastic image of the entire medical image region, and then multiply the elastic image with the aforementioned mask image to obtain the local elastic image. Take an ultrasound device as an example, the ultrasound device supports a general anatomical image imaging examination mode and an elastography examination mode. In the general anatomical image imaging examination mode, the ultrasound device acquires a medical image of the heart region of the examined subject, and segments the heart wall region (mask image) in the medical image, and then switches to the elastography examination mode, wherein the ultrasound device acquires an elastic image of the heart region of the examined subject (such as the existing strain-type ultrasound elastic imaging technology, or shear-wave ultrasound elastic imaging technology), and multiplies the mask image by the elastic image to obtain a local elastic image.

The movement of the heart through each heartbeat is called a cardiac cycle. The cardiac cycle consists of two main phases: systole (ejection of blood) and diastole (filling of blood). During systole, the ventricles contract and expel blood from the heart to the body. After ventricular ejection, the heart enters diastole. In early diastole, the atria are filled with blood returned from the body. The heart then enters a short period of rest called diastole. After diastole, the atria contract, ejecting blood into the ventricles. After atrial contraction, the heart enters the next systolic phase.

In some embodiments, the medical image obtained from current scan as well as the local elastic image may be acquired or generated at any point in the cardiac cycle, e.g., may be acquired or generated in diastole or acquired or generated in systole. Acquisition/generation in diastole or acquisition/generation in systole may be used for different clinical disease diagnoses, which may be determined according to actual requirements.

For example, in some embodiments, it is possible to determine an end of diastole of an examined subject, and to acquire the medical image from the scan at the end of diastole, as well as to determine elastic parameters at the end of diastole of the heart, and generate a local elastic image, which means that the elastic parameters reflect the stiffness of the heart wall region at the end of diastole of the heart. The temporal phase of the cardiac cycle is associated with electrical signals generated by the heart. These electrical signals are usually monitored by an electrocardiogram (ECG). During an ECG, multiple electrodes are placed on the chest and/or extremities to record the electrical signals from the heart. These electrical signals are provided in a visual manner, usually on a monitor, and are provided as ECG traces, with some features associated with specific points in the cardiac cycle. For example, P waves are usually associated with the onset of atrial contractions, while R waves of QRS complex waves are usually associated with the onset of ventricular contractions. Thus, the end of diastole can be determined from the ECG signals, e.g., the end of diastole is the last cardiac phase when triggered on the R wave, and occurs before the R wave of the next cardiac cycle.

In some embodiments, at the end of diastole, the heart tissue does not contract significantly, and therefore does not interfere with the measurement of elastic parameters. In addition, at the end of diastole, the heart is at maximum volume, and a portion of the heart stops briefly. Therefore, acquiring the medical image from the scan at the end of diastole and generating a local elastic image can be used for rapid diagnosis of diseases such as heart attack.

In some embodiments, after a local elastic image is obtained, the local elastic image can be displayed at the position of the heart wall region in the medical image in an overlapping manner and in real-time. For example, the local elastic image is displayed on the medical image (ultrasound B-mode image) at a position corresponding to the heart wall region in an overlapping manner and in real-time. Wherein, the transparency A in the pixel value (ARGB value) of each pixel position in the heart wall region in the local elastic image can be set to a semi-transparent value, so as to be overlaid on the medical image for display.

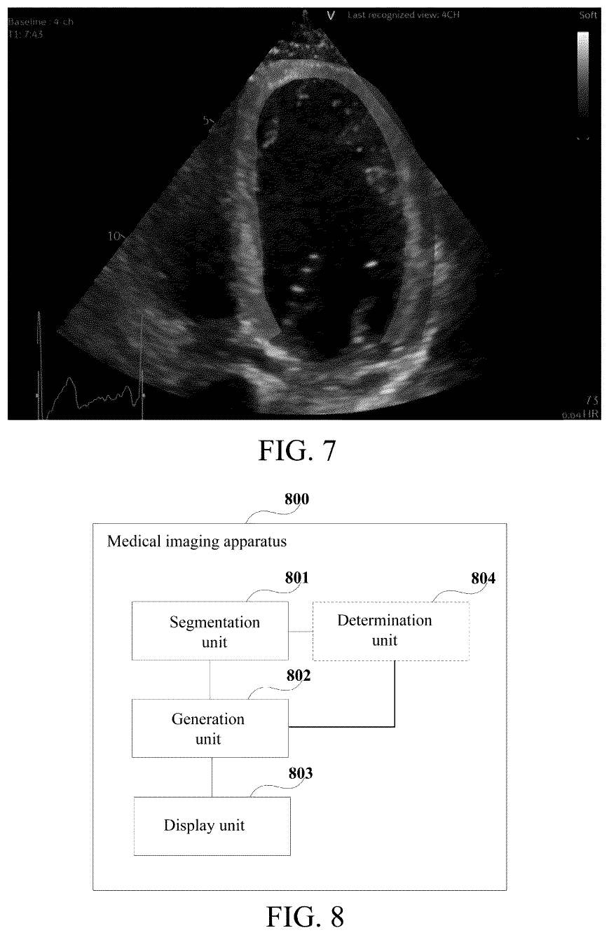

FIG. 7 is a schematic diagram showing a medical image and a local elastic image displayed in an overlapping manner according to an embodiment of the present application. As shown in FIG. 7, the medical image obtained from the current scan and the elastic image of the region of interest in the medical image can be displayed simultaneously in the same image in real-time to facilitate clinical diagnosis. For example, in a healthy state, the left ventricle is elongated and the relative or absolute values of the elastic parameters in the heart wall region (e.g., the myocardial region) are high. However, if the left ventricle is observed to be round and the overall elastic parameters in the heart wall region are low, e.g., if the local elastic image displayed in an overlapping manner on the image displayed in real-time is blue overall, the heart of the examined subject may have a problem with myocardial hypertrophy. In addition, if localized elastic parameters in the heart wall region are observed to be low, e.g., if a localized region (middle or myocardial base) in the local elastic image displayed in an overlapping manner on the image displayed in real-time is blue in color, the heart of the examined subject may have an infarct.

As can be seen from the above embodiments, by segmenting the heart wall region and only displaying elastography in the heart wall region in an overlapping manner and in real-time, the elastography of the heart wall region can thus be more intuitively observed, thereby allowing real-time assessment of myocardial strain, and contributing to rapid clinical diagnosis.

Embodiments of the present invention further provide a medical imaging apparatus, and repetitive contents from the preceding embodiments are not given herein. FIG. 8 is a schematic diagram of a medical imaging apparatus according to an embodiment of the present application, and as shown in FIG. 8, the medical imaging apparatus 800 includes a segmentation unit 801, a generation unit 802, a display unit 803, and a determination unit 804. The segmentation unit 801 is configured to perform image segmentation of a medical image acquired from a current scan containing a heart region of an examined subject to determine a heart wall region in the medical image. The generation unit 802 is configured to generate a local elastic image of the heart wall region. The display unit 803 is configured to display the local elastic image at the position of the heart wall region in the medical image in an overlapping manner and in real-time.

In some embodiments, the medical image is a grayscale image and the local elastic image is a color image. For example, the medical image is an ultrasound B-mode image.

In some embodiments, the segmentation unit 801 uses a deep learning algorithm to perform image segmentation.

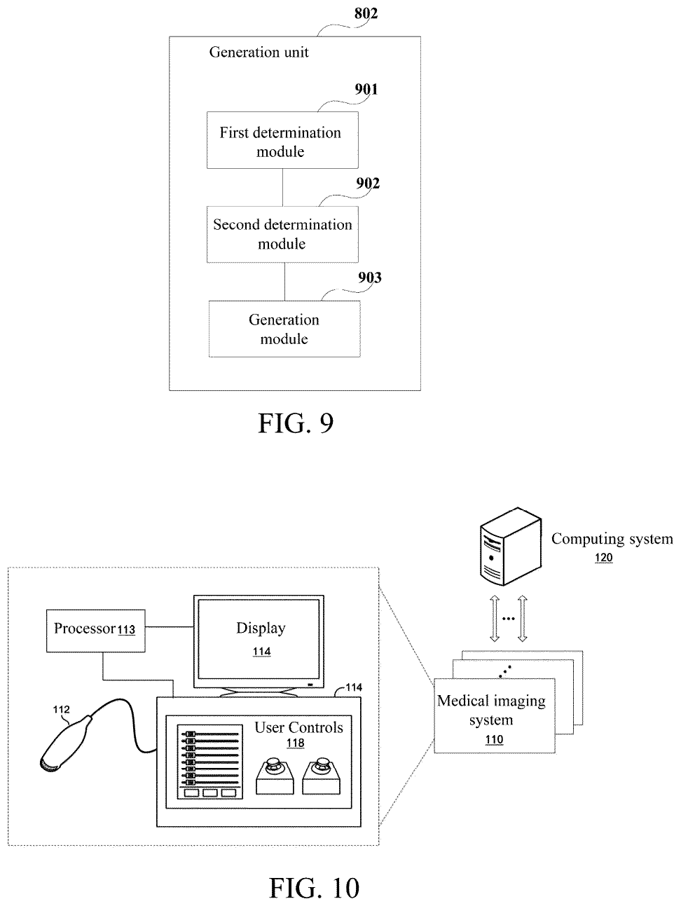

FIG. 9 is a schematic diagram of a generation unit 802 according to an embodiment of the present application, and as shown in FIG. 9, the generation unit 802 includes a first determination module 901, a second determination module 902, and a generation module 903. The first determination module 901 is configured to determine absolute or relative values of elastic parameters at various positions in the heart wall region. The second determination module 902 is configured to determine color codes corresponding to the absolute or relative values of the elastic parameters. The third generation module 903 is configured to generate the local elastic image according to the corresponding color codes at various positions in the heart wall region.

In some embodiments, the elastic parameters are parameters that reflect the stiffness of the tissue organ and include one of Young's modulus, elastic modulus, shear modulus, and shear wave propagation velocity.

In some embodiments, optionally, as shown in FIG. 8, the apparatus may further comprise:

a determination unit 804 which determines an end of diastole of the heart of the examined subject,

the medical image being acquired from the scan at the end of diastole, and the generation unit 802 generating the local elastic image at the end of diastole.

In some embodiments, for the specific implementation methods of the segmentation unit 801, the generation unit 802 and the display unit 803, references can be made to 101-103 in the preceding embodiments, and for the implementation methods of the first determination module 901, the second determination module 902, and the generation module 903, references can be made to 501-503 in the preceding embodiments, and the repetitive contents are not given herein.

In some embodiments, the functions of the segmentation unit 801 and the generation unit 802 may be integrated into a processor for implementation. Wherein, the processor is configured to implement the medical imaging method as described in the preceding embodiments. The processor, which may also be referred to as a microcontroller unit (MCU), microprocessor, or microcontroller or other processor devices and/or logic devices, may include reset circuitry, clock circuitry, chips, microcontrollers, and so on. The functions of the processor may be integrated on the main board of the medical device (e.g., the processor is configured as a chip connected to the main board processor (CPU)), or may be configured independently of the main board, and embodiments of the present invention are not limited thereto.

As can be seen from the above embodiments, by segmenting the heart wall region and only displaying elastography in the heart wall region in an overlapping manner and in real-time, the elastography of the heart wall region can thus be more intuitively observed, thereby allowing real-time assessment of myocardial strain, and contributing to rapid clinical diagnosis.

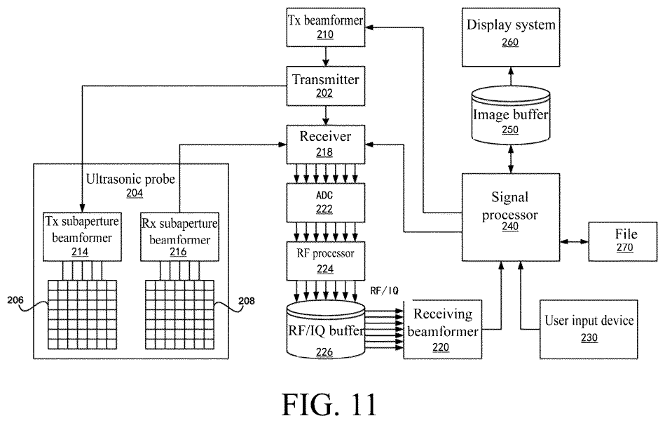

Embodiments of the present invention further provide a medical imaging system, and FIG. 10 is a schematic diagram of a medical imaging system according to an embodiment of the present application, and as shown in FIG. 10, the medical imaging system 110 includes suitable hardware, software, or a combination thereof for supporting medical imaging (i.e., enabling the acquisition of data for use in generating and/or rendering images during a medical imaging examination). For example, the medical imaging system 110 may be an ultrasound system or magnetic resonance system configured to generate and/or render ultrasound images, etc. FIG. 11 depicts an illustrative specific implementation of an ultrasound system that may correspond to the medical imaging system 110, and detailed illustration will be provided below. As shown in FIG. 10, the medical imaging system 110 may include a scan device 112, a display 114, and a processor 113, and the scan device may be portable and movable.

The scan device 112 may be configured to generate and/or capture specific types of imaging signals (and/or data corresponding thereto), e.g., by moving over the examined subject (or a portion thereof), and may include suitable circuitry for performing and/or supporting such functions. The scan device 112 may be an ultrasonic probe, an MRI scanner, a CT scanner, or any suitable imaging device. For example, in the case where the medical imaging system 110 is an ultrasound system, the scan device 112 may emit an ultrasound signal and capture an echo ultrasound image.

The display 114 may be configured to display images (e.g., via a screen). In some cases, the display 114 may also be configured to at least partially generate the displayed image. In addition, the display 114 may further support user input/output. For example, in addition to images, the display 114 may further provide (e.g., via the screen) user feedback (e.g., information related to the system, the functions, the settings thereof, etc.). The display 114 may further support user input (e.g., via user controls 118) to, for example, allow control of medical imaging. User input can involve controlling the display of images, selecting settings, specifying user preferences, requesting feedback, etc.

In some embodiments, the medical imaging system 110 may further incorporate additional and dedicated computing resources, such as one or more computing systems 120. In this regard, each computing system 120 may include suitable circuitry, interfaces, logic, and/or code for processing, storing, and/or communicating data. The computing system 120 may be a specialized device configured for use specifically in conjunction with medical imaging, or it may be a general-purpose computing system (e.g., a personal computer, server, etc.) that is set up and/or configured to perform the operations described below with respect to the computing system 120. The computing system 120 may be configured to support the operation of the medical imaging system 110, as described below. In this regard, various functions and/or operations can be offloaded from the imaging system. Doing so can simplify and/or centralize certain aspects of processing to reduce costs (by eliminating the need to add processing resources to the imaging system).

The computing system 120 may be set up and/or arranged for use in different ways. For example, in some specific implementations, a single computing system 120 may be used, and in other specific implementations, multiple computing systems 120 are configured to work together (e.g., based on a distributed processing configuration), or individually, wherein each computing system 120 is configured to process specific aspects and/or functions, and/or to process data only for a specific medical imaging system 110.

In some embodiments, the computing system 120 may be local (e.g., co-located with one or more medical imaging systems 110, such as within the same facility and/or the same local network); and in other specific embodiments, the computing system 120 may be remote, and thus accessible only via a remote connection (e.g., via the Internet or other available remote access technology). In particular specific implementations, the computing system 120 may be configured in a cloud-based manner and may be accessed and/or used in a substantially similar manner to accessing and using other cloud-based systems.

Once the data is generated and/or configured in the computing system 120, the data can be copied and/or loaded into the medical imaging system 110. This can be done in different ways. For example, data may be loaded via a directed connection or link between the medical imaging system 110 and the computing system 120. In this regard, communication between the different components of the setup can be performed using available wired and/or wireless connections and/or according to any suitable communication (and/or networking) standards or protocols. Optionally or additionally, the data may be loaded indirectly into the medical imaging system 110. For example, data may be stored in a suitable machine-readable medium (e.g., flash memory card, etc.) and then loaded into the medical imaging system 110 using the machine-readable medium (on-site, such as by a user of the system (e.g., imaging clinician) or authorized personnel); or the data may be downloaded to a locally communicative electronic device (e.g., laptop, etc.) and then such electronic device is used on-site (e.g., by a user of the system or authorized personnel) to upload the data to the medical imaging system 110 via a direct connection (e.g., USB connector, etc.).

In operation, the medical imaging system 110 may be used to generate and present (e.g., render or display) images during a medical examination and/or used in conjunction therewith to support user input/output. The images can be 2D, 3D, and/or 4D images. The particular operations or functions performed in the medical imaging system 110 to facilitate the generation and/or presentation of images depend on the type of system (i.e., the means used to obtain and/or generate the data corresponding to the images). For example, in ultrasound imaging, the data are based on the emitted ultrasound signal and the echo ultrasound signal, as described in more detail with respect to FIG. 11.

In some embodiments, the scan device 112 scans a heart region of an examined subject during a general anatomical image imaging examination to obtain imaging data. The processor 113 generates a medical image containing the heart region of the examined subject according to said imaging data. The display 114 may display the medical image generated based on the currently acquired imaging data in real-time. The processor 113 performs image segmentation of said medical image to determine a heart wall region in said medical image. The scan device 112 then scans the heart region during the elastography examination to obtain elastography data, and the processor 113 generates a local elastic image of the heart wall region based on the elastography data. Specific implementation methods are as previously described. The display 114 displays said local elastic image at the position of said heart wall region in said medical image in an overlapping manner and in real-time.

FIG. 11 is a schematic diagram of an ultrasound imaging system according to an embodiment of the present application, and as shown in FIG. 11, the ultrasound system 200 may be configured to provide ultrasound imaging, and may therefore include suitable circuitry, interfaces, logic, and/or code for performing and/or supporting ultrasound imaging related functions. The ultrasound system 200 may correspond to the medical imaging system 110 of FIG. 10.

The ultrasound system 200 includes, for example, a transmitter 202, an ultrasonic probe 204 (scan device), a transmitting beamformer 210, a receiver 218, a receiving beamformer 220, an RF processor 224, an RF/IQ buffer 226, a user input module 230, a signal processor 240 (processor), an image buffer 250, a display system 260 (display), and a file 270.

The transmitter 202 may include suitable circuitry, interfaces, logic, and/or code operable to drive the ultrasonic probe 204. The ultrasonic probe 204 may include an array of two-dimensional (2D) piezoelectric elements. The ultrasonic probe 204 may include a set of transmitting transducer elements 206 and a set of receiving transducer elements 208 that typically form the same element. In some embodiments, the ultrasonic probe 204 may be operable to acquire ultrasound image data covering at least a substantial portion of an anatomical structure (such as the heart or any suitable anatomical structure).

The transmitting beamformer 210 may include suitable circuitry, interfaces, logic, and/or code that is operable to control the transmitter 202, and the transmitter 202 drives the set of transmitting transducer elements 206 through a transmitting subaperture beamformer 214 to transmit ultrasound emission signals into a region of interest (e.g., a person, animal, subsurface cavity, physical structure, etc.). The emitted ultrasound signal can be backscattered from structures in the subject of interest (e.g., blood cells or tissue) to produce echoes. The echo is received by the receiving transducer element 208.

The set of receiving transducer elements 208 in the ultrasonic probe 204 may be operated to convert the received echo to an analog signal for subaperture beam formation through a receiving subaperture beamformer 216, which is then transmitted to the receiver 218. The receiver 218 may include suitable circuitry, interfaces, logic, and/or code that is operable to receive signals from the receiving subaperture beamformer 216. The analog signal can be transferred to one or more of multiple A/D converters 222.

The plurality of A/D converters 222 may include suitable circuitry, interfaces, logic, and/or code that is operable to convert the analog signal from the receiver 218 to a corresponding digital signal. A plurality of A/D converters 222 are provided between the receiver 218 and the RF processor 224. Nevertheless, the present disclosure is not limited in this regard. Thus, in some embodiments, a plurality of A/D converters 222 may be integrated within the receiver 218.

The RF processor 224 may include suitable circuitry, interfaces, logic, and/or code that is operable to demodulate the digital signals output by the plurality of A/D converters 222. According to one embodiment, the RF processor 224 may include a complex demodulator (not shown) that is operable to demodulate the digital signal to form an I/Q data pair representing the corresponding echo signal. The RF or I/Q signal data can then be transferred to the RF/IQ buffer 226. The RF/IQ buffer 226 may include suitable circuitry, interfaces, logic, and/or code that is operable to provide temporary storage of RF or I/Q signal data generated by the RF processor 224.

The receiving beamformer 220 may include suitable circuitry, interfaces, logic, and/or code that may be operable to perform digital beamforming processing to, for example, sum and output a beam summing signal for the delay-channel signals received from the RF processor 224 via the RF/IQ buffer 226. The resulting processed information may be the beam summing signal output from the receiving beamformer 220 and transmitted to the signal processor 240. According to some embodiments, the receiver 218, a plurality of A/D converters 222, the RF processor 224, and the beamformer 220 may be integrated into a single beamformer which may be digital. In various embodiments, the ultrasound system 200 includes a plurality of receiving beamformers 220.

The user input device 230 can be used to enter patient data, scan parameters, and settings, and select protocols and/or templates to interact with the AI segmentation processor, so as to select tracking targets, etc. In an illustrative embodiment, the user input device 230 is operable to configure, manage, and/or control the operation of one or more components and/or modules in the ultrasound system 200. In this regard, the user input device 230 is operable to configure, manage, and/or control the operation of the transmitter 202, the ultrasonic probe 204, the transmitting beamformer 210, the receiver 218, the receiving beamformer 220, the RF processor 224, the RF/IQ buffer 226, the user input device 230, the signal processor 240, the image buffer 250, the display system 260, and/or the file 270.

For example, the user input devices 230 may include buttons, rotary encoders, touch screens, motion tracking, voice recognition, mouse devices, keyboards, trackballs, cameras, and/or any other devices capable of receiving user commands. In some embodiments, for example, one or more of the user input devices 230 may be integrated into other components (such as the display system 260 or the ultrasonic probe 204). As an example, the user input device 230 may include a touch screen display. As another example, the user input device 230 may include an accelerometer, gyroscope, and/or magnetometer attached to and/or integrated with the probe 204 to provide pose and motion recognition of the probe 204, such as identifying one or more probe compressions against the patient's body, predefined probe movements, or tilt operations, etc. Additionally and/or alternatively, the user input device 230 may include image analysis processing to identify the probe pose by analyzing the captured image data.

The signal processor 240 may include suitable circuitry, interfaces, logic, and/or code that is operable to process the ultrasound scan data (i.e., the summed IQ signal) to generate an ultrasound image for presentation on the display system 260. The signal processor 240 is operable to perform one or more processing operations based on a plurality of selectable ultrasound modalities on the acquired ultrasound scan data. In an illustrative embodiment, the signal processor 240 is operable to perform display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various embodiments, the processed image data may be presented at the display system 260 and/or may be stored in the file 270. The file 270 can be a local file, a picture archiving and communication system (PACS), or any suitable device for storing images and related information.

The signal processor 240 may be one or more central processing units, microprocessors, microcontrollers, etc. For example, the signal processor 240 may be an integrated component, or may be distributed in various locations. The signal processor 240 may be configured to receive input information from the user input device 230 and/or file 270, generate outputs that may be displayed by the display system 260, and manipulate the outputs, etc., in response to the input information from the user input device 230. The signal processor 240 may be capable of executing, for example, any of one or more of the methods and/or one or more sets of instructions discussed herein according to various embodiments.

The ultrasound system 200 may be operated to continuously acquire ultrasound scan data at a frame rate suitable for the imaging situation under consideration. Typical frame rates are in the range of 20 to 220, but can be lower or higher. The acquired ultrasound scan data can be shown on the display system 260 in real-time at a display rate that is the same as the frame rate, or slower, or faster than the frame rate. The image buffer 250 is included to store frames for processing of the acquired ultrasound scan data that are not scheduled for immediate display. Preferably, the image buffer 250 has sufficient capacity to store frames of ultrasound scan data for at least a few minutes. Frames of ultrasound scan data are stored in such a way that they can be easily retrieved therefrom according to their acquisition sequence or time. The image buffer 250 may be embodied in any known data storage medium.

In some specific embodiments, the signal processor 240 may be configured to perform or otherwise control at least some of the functions performed thereby based on user instructions via the user input device 230. As an example, the user may provide voice commands, probe poses, button presses, etc. to issue specific commands such as controlling aspects of automatic strain measurement and strain ratio calculations, and/or provide or otherwise specify various parameters or settings associated therewith, as described in more detail below.

In operation, the ultrasound system 200 may be used to generate ultrasound images, including two-dimensional (2D), three-dimensional (3D), and/or four-dimensional (4D) images. In this regard, the ultrasound system 200 is operable to continuously acquire ultrasound scan data at a specific frame rate, which may be applicable to the imaging situation discussed. For example, the frame rate can be in the range of 20-70, or can be lower or higher. The acquired ultrasound scan data can be shown on the display system 260 at the same display rate as the frame rate, or slower, or faster than the frame rate. The image buffer 250 is included to store frames for processing of the acquired ultrasound scan data that are not scheduled for immediate display. Preferably, the image buffer 250 has sufficient capacity to store at least a few seconds of frames of ultrasound scan data. Frames of ultrasound scan data are stored in such a way that they can be easily retrieved therefrom according to their acquisition sequence or time. The image buffer 250 may be embodied in any known data storage medium.

In some cases, the ultrasound system 200 may be configured to support grayscale and color-based operations. For example, the signal processor 240 is operable to perform grayscale B-model processing and/or color processing. The grayscale B-model processing may include processing B-model RF signal data or IQ data pairs. For example, the grayscale B-model processing can enable the formation of an envelope of the received beam summing signal by computing the amount (I²±Q²)^{1/2}. The envelope can be subjected to additional B-model processing, such as logarithmic compression to form the display data. The display data can be converted to X-Y format for video display. Scan-converted frames can be mapped to grayscale for display. The B-model frame is provided to the image buffer 250 and/or the display system 260. Color processing may include processing color-based RF signal data or IQ data pairs to form frames to cover the B-model frames being provided to image buffer 250 and/or the display system 260. Grayscale and/or color processing may be self-adaptively adjusted based on user input (e.g., selections from the user input device 230), such as for enhancing the grayscale and/or color of a particular region.

In some embodiments, the ultrasonic probe 204 scans a heart region of an examined subject during a general anatomical image imaging examination. The receiver 218 acquires imaging data. The signal processor 240 generates a medical image (ultrasound B -mode image) containing the heart region of the examined subject according to the imaging data. The display system 260 may display in real-time the medical image (ultrasound B-mode image) generated based on the currently acquired imaging data. The signal processor 240 (the neural network model therein) performs image segmentation of the medical image to determine a heart wall region in the medical image. The ultrasonic probe 204 then scans (presses or tracks shear waves) the heart region during the elastography examination. The signal processor 240 determines the elastography data (absolute or relative values of elastic parameters), and generates a local elastic image of the heart wall region according to the elastography data. The specific implementation method is as described previously. The display system 260 displays the local elastic image at the position of the heart wall region in the medical image in an overlapping manner and in real-time.

Embodiments of the present invention further provide a computer readable program, wherein upon execution of said program, said program causes the computer to perform the medical imaging method described in the preceding embodiments in said device, or system, or medical device.

Embodiments of the present invention further provide a storage medium storing a computer readable program, wherein said computer readable program causes a computer to perform the medical imaging method described in the preceding embodiments in a device, or system, or medical device.

The above embodiments merely provide illustrative descriptions of the embodiments of the present application. However, the present application is not limited thereto, and appropriate variations may be made on the basis of the above embodiments. For example, each of the above embodiments may be used independently, or one or more of the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A medical imaging method, comprising:
performing image segmentation of a medical image acquired from a current scan containing a heart region of an examined subject to determine a heart wall region in said medical image; and
generating a local elastic image of said heart wall region, and displaying said local elastic image at the position of said heart wall region in said medical image in an overlapping manner and in real-time, wherein the local elastic image of the heart wall region is displayed only at the position of said heart wall region determined by the image segmentation.

2. The method according to claim 1, wherein said medical image is a grayscale image and said local elastic image is a color image.

3. The method according to claim 1, wherein said medical image is an ultrasound B -mode image.

4. The method according to claim 1, wherein the performing image segmentation comprises: performing image segmentation using a deep learning algorithm or a machine learning algorithm.

5. The method according to claim 1, wherein the generating a local elastic image of said heart wall region comprises:
determining absolute or relative values of elastic parameters at various positions in said heart wall region;
determining color codes corresponding to the absolute or relative values of said elastic parameters; and
generating said local elastic image according to the corresponding color codes at various positions in said heart wall region.

6. The method according to claim 5, wherein said elastic parameter is a parameter reflecting the stiffness of a tissue organ, including one of Young's modulus, elastic modulus, shear modulus, and shear wave propagation velocity.

7. The method according to claim 1, further comprising:
determining an end of diastole of the heart of said examined subject;
and, acquiring said medical image from the scan at said end of diastole, as well as generating said local elastic image at said end of diastole.

8. A medical imaging apparatus, comprising:
a segmentation unit which performs image segmentation of a medical image acquired from a current scan containing a heart region of an examined subject to determine a heart wall region in said medical image;
a generation unit which generates a local elastic image of said heart wall region; and
a display unit which displays said local elastic image at the position of said heart wall region in said medical image in an overlapping manner and in real-time, wherein the local elastic image of the heart wall region is displayed only at the position of said heart wall region determined by the image segmentation.

9. The apparatus according to claim 8, wherein said medical image is a grayscale image and said local elastic image is a color image.

10. The apparatus according to claim 8, wherein said medical image is an ultrasound B-mode image.

11. The apparatus according to claim 8, wherein said segmentation unit uses a deep learning algorithm or a machine learning algorithm to perform image segmentation.

12. The apparatus according to claim 8, wherein said generation unit comprises:
a first determination module which determines absolute or relative values of elastic parameters at various positions in said heart wall region;
a second determination module which determines color codes corresponding to the absolute or relative values of said elastic parameters; and
a generation module which generates said local elastic image according to the corresponding color codes at various positions in said heart wall region.

13. The apparatus according to claim 12, wherein said elastic parameter is a parameter reflecting the stiffness of a tissue organ, including one of Young's modulus, elastic modulus, shear modulus, and shear wave propagation velocity.

14. The apparatus according to claim 8, further comprising:
a determination unit which determines an end of diastole of the heart of said examined subject,
said medical image being acquired from the scan at said end of diastole, and said generation unit generating said local elastic image at said end of diastole.

15. A medical imaging system, comprising:
a scan device which is used to scan a heart region of an examined subject to obtain imaging data;
a processor which is configured for generating a medical image containing the heart region of the examined subject according to said imaging data, performing image segmentation of said medical image to determine a heart wall region in said medical image, and generating a local elastic image of said heart wall region; and
a display which displays said local elastic image at the position of said heart wall region in said medical image in an overlapping manner and in real-time, wherein the local elastic image of the heart wall region is displayed only at the position of said heart wall region determined by the image segmentation.

16. The system according to claim 15, wherein said medical image is a grayscale image and said local elastic image is a color image.

17. The system according to claim 15, wherein the performing image segmentation comprises: performing image segmentation using a deep learning algorithm or a machine learning algorithm.

18. The system according to claim 15, wherein the generating a local elastic image of said heart wall region comprises:
determining absolute or relative values of elastic parameters at various positions in said heart wall region;
determining color codes corresponding to the absolute or relative values of said elastic parameters; and
generating said local elastic image according to the corresponding color codes at various positions in said heart wall region.

19. The system according to claim 18, wherein said elastic parameter is a parameter reflecting the stiffness of a tissue organ, including one of Young's modulus, elastic modulus, shear modulus, and shear wave propagation velocity.

20. The system according to claim 15, wherein said processor is further configured for:
determining an end of diastole of the heart of said examined subject;
and, acquiring said medical image from the scan at said end of diastole, as well as generating said local elastic image at said end of diastole.
